(19) United States
(12) Patent Application Publication
(10) Pub. No.: US 2016/0331463 A1
(43) Pub. Date: Nov. 17, 2016

(54) METHOD FOR GENERATING A 3D REFERENCE COMPUTER MODEL OF AT LEAST ONE ANATOMICAL STRUCTURE

(71) Applicant: AO TECHNOLOGY AG, Chur (CH)

(72) Inventors: Christoph Nötzli, Davos Platz (CH); Lukas Kamer, Schindellegi (CH)

(21) Appl. No.: 15/110,735

(22) PCT Filed: Jan. 10, 2014
(86) PCT No.: PCT/CH2014/000003

§ 371 Date: Jul. 9, 2016

(51) Int. Cl. A61B 34/10; G06T 19/20; G06T 7/00; G06T 17/00

(52) U.S. Cl. CPC A61B 34/10; G06T 17/00; G06T 19/20; G06T 7/0032; G06T 2210/41; G06T 2207/30008; G06T 2207/20101; G06T 2207/20104; G06T 2207/10004; A61B 2034/101

(57) ABSTRACT

A method for generating a 3D reference computer model of at least one anatomical structure for comparison with a selectable pre-, intra- or postoperative set of medical images of at least one anatomical structure, the method including: acquiring at least a first and a second medical image of at least one anatomical structure in a preoperative status from different perspectives using a computer assisted medical imaging device, wherein the first and second medical images are represented by a respective first and second set of digital 2D image data; and generating a 3D reference computer model of an anatomical structure by selecting and extracting a 3D atlas model of an anatomical structure to be treated from a generic anatomical atlas provided in the form of a digital data source, and registering at least a section of each of the first and second medical images to the selected 3D atlas model.

### BACKGROUND OF THE INVENTION

1. Field of the Invention

The invention relates to a method for generating a 3D reference computer model of at least one anatomical structure according to the preamble of claim 1, to a method for generating a status related 3D computer model of a patient's anatomical structure in a pre-, intra- or post-operative status according to the preamble of claim 10 or 11 and to a method for monitoring a surgical treatment according to the preamble of claim 26.

During surgical treatments of fractures and the correction of osseous deformities bone fragments are anatomically repositioned and stably fixed at a correct position by using suitable fixation techniques. Problems may arise by an unrecognized malposition of bone fragments and implants during surgery, or through their secondary dislocation in the postoperative course. A faulty osteosynthesis due to anatomically incorrect repositioning of bone fragments, improper surgical technique, unsuitable selection of an implant and/or its positioning is to be avoided.

Bone fractures and osseous deformities are routinely assessed using different radiological imaging techniques before, during, and after surgery. Usually conventional x-rays are used, i.e. planar projection images. Particularly complex interventions are assessed for diagnostic purposes by using a tomographic layer imaging, preferably by using computer tomography (CT). This is done by analyzing these layer images or their three-dimensional computer models preferably preoperatively, in the case of special issues also intra- or post-operatively.

However, so far in clinical routine the bone fragments and the osteosynthesis cannot be assessed spatially coherent over the entire course of therapy. Three-dimensional medical imaging as CT's in all stages of therapy would be needed. As mentioned this is technically possible, but so far costs, radiation-hygienic reasons, generation of artifacts, personal, organizational and technical effort clearly oppose a routine spatial assessment of osteosynthesis in all stages of therapy.

2. Description of the Related Art

A process for the reduction of fragments of a fractured bone is known from US-A 2011/0082367 REGAZZONI. This known process includes steps of generating 3D representations of bones and bone fragments on the basis of a digital data set obtained by means of CT's of a fractured bone, as well as of the contralateral healthy bone of a patient. The 3D representation of the mirrored contralateral healthy bone is used as a reference model for the relative position of the 3D representations of repositioned bone fragments. Subsequently, the 3D representations of the proximal and distal bone fragments are matched with the 3D representation of the reference model using three-dimensional image registration. Furthermore, the configurations of markers and/or anatomical landmarks on the proximal and the distal bone fragment are extracted and transferred to the reference model. The relative positions of the markers and/or anatomical markers transferred to the reference model of the proximal and distal bone fragments then allow to establish a digital reference data set suitable for the real reduction of the bone fragments during the operation. A disadvantage of this known method can be that each a CT of the fractured bone and of the contralateral healthy bone is needed.

### BRIEF SUMMARY OF THE INVENTION

It is therefore an object of the invention to provide a method for generating a 3D reference computer model of at least one anatomical structure which requires an acquisition of standard 2D medical images only.

The invention solves the posed problem with a method for generating a 3D reference computer model of at least one anatomical structure comprising the features of claim 1, with a method for generating a status related 3D computer model of a patient's anatomical structure in a pre-, intra- or post-operative status comprising the features of claim 10 or 11 and to a method for monitoring a surgical treatment comprising the features of claim 26.

The advantages of the method according to the invention can essentially be seen in:
a full 3D computer model of a patient affected by a bone fracture or bone deformity can be established from conventional 2D medical images only. This permits the patient to be assessed and its treatment to be guided and tracked in 3D; and
less image information is needed to get comprehensive information to assess the surgical treatment of the patient at any stage.

Certain terms as used herein are understood as follows:

### 3D Reference Computer Model:

A full body 3D atlas model 30 with standard deviation information is superposed on the first and second medical images (preferably medical 2D images) of a given clinical case and they are referenced on the 3D atlas model, by the specific values gathered in predefined well detectable zones and/or artificial additional markers outside and/or anatomical landmarks inside the body. The first and second medical images might be taken, with different existing and new technologies/modalities (e.g. known X-ray techniques or CT-scans) to allow differentiating several independent, but known values, respectively value maps, which are an integrated part of the full body 3D atlas model. Differences of the first and second medical images (the individual 2D images) to the 3D atlas model are first used in an analysis using only healthy information like landmarks of unfractured bone to adapt the 3D atlas model to the first and second medical images of the individual case and fill up the gaps of information, such that the “normal” 3D atlas model is converted to the individual measures and by this transformed in a 3D reference computer model, i.e. in a full 3D redesign of the individual healthy body.

### Status Related 3D Computer Model:

By superposing the 3D reference computer model on the first and second medical images additional variations especially from the pathological area are detected either as deformities or as fragments in dislocation. By using this technology the 3D reference computer model as a full 3D model of the healthy situation can be transformed to a corresponding status related 3D computer model, e.g. a pathological 3D model.

Alternatively or additionally, by superposing the 3D reference computer model on subsequent pre-, intra- or post-operative sets of medical images the 3D reference computer model as a full 3D model of the healthy situation can be transformed to a corresponding status related 3D computer model in a pre-, intra- or postoperative status i.e. to any pathological or surgically treated 3D redesign at any stage of healing.

### Graphical 3D Computer Model:

The graphical 3D computer model includes computer-aided planning and performing a virtual surgical treatment of anatomical structures to be treated by using the 3D reference computer model and/or the pre-operative status related 3D computer model.

### Implant:

The term implant as used herein is understood as including all solid means artificially implanted or to be implanted in the human or animal body completely or partially which can be detected by conventional x-rays, CT or magnetic resonance imaging (MRI) and which have a limited variability in their form, such as orthopedic implants, dental implants, pacemakers or stents.

### Registration:

Image registration is understood as the process of mapping one or more target images of an object to a reference image, thereby establishing point-by-point correspondence between the reference image and the target image. The step “registering” preferably comprises the following sub-steps (B. Zitova, J. Flusser, Image registration methods: a survey, Image and Vision Computing 21, 2003, 977-1000):
1) Feature detection: salient and distinctive objects (closed boundary regions, edges, contours, line intersections, corners etc.) are manually or, preferably automatically detected. For further processing these features can be represented by their point representatives (centers of gravity, line endings, distinctive points);
2) Feature matching: in this step the correspondence between the features detected in the sensed image (3D atlas model 30) and those detected in the reference image (first and second medical images 10, 11) is established. Various feature descriptors and similarity measures along with spatial relationships among the features are used for that purpose;
3) Transform model estimation: the type and parameters of the so-called mapping functions, aligning the sensed image with the reference image, are estimated. The parameters of the mapping functions are computed by means of the established feature correspondence; and
4) Image resampling and transformation: the sensed image (3D atlas model 30) is transformed by means of the mapping functions. By means of the mapping function the sensed image (3D atlas model 30) is transformed to overlay it over the reference image (first and second medical images 10, 11).

The above sub-steps are used herein for the case of a “scene to model registration” where the images of a scene (anatomy of the patient) and a model of the scene (3D atlas model) are registered.

Further advantageous embodiments of the invention can be commented as follows:

In a special embodiment the first and second medical images are taken from different perspectives that are minimum 60° angularly offset with respect to each other.

In a further embodiment the at least one anatomical structure is a bone and the registering step includes before performing the image registration the sub-steps of: extracting a first section of the first medical image, wherein the first section of the first medical image comprises a section of a proximal bone fragment spaced apart from a fracture site or from a deformed portion of a bone; extracting a second section of the first medical image, wherein the second section of the first medical image comprises a section of a distal bone fragment spaced apart from a fracture site or from a deformed portion of a bone; and repeating the above steps for the second medical image.

In a further embodiment the first and second medical images include a plurality of anatomical structures and the 3D reference computer model comprises each a graphical 3D sub-model for each anatomical structure. An advantage achieved by this means is, that individually trackable graphical 3D sub-models for the anatomical structures to be treated like bones or bone fragments can be integrated in the 3D reference computer model allowing an individual analysis of certain anatomical structures.

In another embodiment the method further comprises the additional step of: introducing at least one digital graphical 3D sub-model in the 3D reference computer model. A graphical 3D sub-model of an implant and/or of a surgical instrument can be copied from a database in the 3D reference computer model, such as for example a CAD database.

In a further embodiment the digital graphical 3D sub-model represents an implant.

In again another embodiment the digital graphical 3D sub-model represents a surgical instrument.

In a further embodiment the generation of the 3D reference computer model comprises an automatic or manual identification and localization of anatomical landmarks, lines and/or regions of the anatomical structures to be treated.

In a further embodiment the generation of the 3D reference computer model comprises an automatic or manual identification and localization of distinctive points, lines and/or regions of each implant and preferably of each surgical instrument.

The method for generating a status related 3D computer model of a patient's anatomical structure in the pre-operative status by using the 3D reference computer model comprises the step of: registering each of the first and second medical images to the 3D reference computer model. By subsequently superposing the 3D reference computer model on the first and second medical images additional variations especially from the pathological area are detected either as deformities or as fragments in dislocation.

For subsequent status related 3D computer models of a patient's anatomical structure in a pre-, intra- or post-operative status the following steps are performed: a) acquiring a pre-, intra- or post-operative set of medical images including at least two medical images of at least one anatomical structure in a pre-, intra- or post-operative status and from different perspectives by using a computer assisted medical imaging device, wherein the at least two medical images are each represented by a respective set of digital 2D image data; b) generating a graphical 2D or 3D computer model of at least one anatomical structure in the form of a set of digital data by using the pre-, intra- or post-operative medical images; and c) registering the graphical 2D or 3D computer model to the 3D reference computer model. The advantages achieved are that due to the registration of conventional preoperative x-rays, intraoperative 2D planar or spatial 3D C-arm images, or postoperative X-ray images to the initially generated 3D reference computer model of anatomical structures (e.g. a bone or bone fragment) these pre-, intra- or postoperatively acquired sets of medical images can now always be represented as status related 3D computer models over the entire course of therapy.

A spatial representation preoperatively generated once and preferably by using a CT is beneficial for several reasons: it generates a spatial representation of the region to be treated at the beginning of the therapy. This spatial information can be used for diagnostics and therapy planning. In addition, preoperatively there is more time available for their processing and analysis as for example during the operation. Further, other imaging techniques, generated by using intraoperative 2D or 3D C-arm images are less or even inappropriate for temporal or technical reasons, to generate 3D computer models of anatomical structures such as bone. The same applies to conventional preoperative and postoperative x-rays, where the scaled representation of 3D computer models of anatomical structures such as bone fragments is not possible; at least not without considerable additional effort. These X-ray images represent planar images, generated from one direction of projection only. But their high image resolution is beneficial.

In a special embodiment the status related 3D computer model additionally comprises a representation of at least one implant.

In a further embodiment the status related 3D computer model additionally comprises a representation of at least one surgical instrument.

In another embodiment the pre-, intra- or post-operative set of medical images includes a plurality of anatomical structures and the status related the 3D computer model comprises each a graphical 2D or 3D sub-model for each anatomical structure and preferably for each implant and/or surgical instrument.

In another embodiment the status related 3D computer model forms the reference model to which the 3D reference computer model is adapted during the registration step. The status related 3D computer model is used as a target model, to which the 3D reference computer model (object model or source model) is modified. The acquisition of the pre-, intra- or postoperative sets of medical images can include two or more digital medical images, which are obtained each at a predefined angle of the image plane of the C-arm with respect to the gravity vector so that the positions of anatomical structures to be treated and hence the position of the 3D reference computer model are defined in a system of coordinates which is fixed with respect to the operation room.

In again another embodiment the acquisition of the set of medical images—in a pre-, intra- or postoperative status—includes an acquisition of one or more digitized medical images by means of a computer-aided medical imaging technique. The acquisition of two or more digitized medical images is performed at an angle relative to each other permits to generate a 3D computer model. On the other hand, different fragments/sections of a long bone can be mapped in each of the digitized medical images so that intra-operatively used C-arm equipment with a relatively small image frame can be used to acquire the pre-, intra- or postoperative sets of medical images. The procedure distinguishes itself by the fact that only one X-ray can be sufficient and standard image acquisitions “in two planes” as known to the skilled person can be avoided. Additional advantages of the method are thus a reduced radiation exposure and expenditure. In the case of corrective osteotomies and fracture treatments the entire osteosynthesis construct consisting of bone fragments, any residual bone defect and the implants used can be spatially assessed over the entire course of therapy. On the computer display a graphical representation of the status related 3D computer model of the anatomical structure such as the fracture or osteotomy is visible, representing spatially the bone fragments depending on the stage of therapy before, during or after surgery. Thus, a 3D imaging procedure is not necessary. As soon as implant material is radiologically visible, its position can also be spatially determined and represented by referencing its 3D computer model to the 3D computer models of anatomical structures, such as for example the bone fragments.

In a further embodiment the generation of the status related 3D computer model includes an automatic or manual re-identification and re-localization of the anatomical landmarks, lines and/or regions of the anatomical structures to be treated as identified and localized in the 3D reference computer model. In the simplest case, the status related 3D computer model is based on a single digital medical image with the re-identified and re-localized anatomical landmarks. The registration can therefore be effected with a feature-based registration process. In the case of feature-based registration processes, a certain, usually relatively small number of features, e.g. anatomical landmarks are extracted from the images. This is done either manually or automatically. The selected anatomical features are preferably spread over the whole image and do not only focus on a single region. The registration is then effected by matching the selected features, e.g. the selected anatomical landmarks on the source model, i.e. the 3D reference computer model with the identical anatomical landmarks on the reference or target model, i.e. on the status related 3D computer model. In addition to anatomical landmarks regions in the image that clearly distinguish from adjacent regions, can be used as region features or lines or edges, which are present as lines or contours of regions can be used as features. Lines can be represented and extracted by their endpoints as well.

In a further embodiment the generation of the status related 3D computer model includes an automatic or manual re-identification and re-localization of the distinctive points, lines and/or regions of each implant and each surgical instrument as identified and localized in the 3D reference computer model. The registration of the 3D sub-models of implants or surgical instruments can be effected in two ways:

(1) first, graphical 3D sub-models of anatomical structures of the 3D reference computer model are registered to the graphical 3D sub-models of the anatomical structures of the status related 3D computer model and subsequently the graphical 3D sub-models of implants or surgical instruments of the 3D reference computer model are registered with one or more graphical 3D sub-models of anatomical structures of the previously registered graphical 3D sub-models of anatomical structures of the 3D reference computer model by thereby taking into consideration the relative positions between the graphical 3D sub-models of implants or surgical instruments and the graphical 3D sub-models of the anatomical structures in the status related 3D computer; or

(2) first, graphical 3D sub-models of anatomical structures of the 3D reference computer model are registered to the graphical 3D sub-models of the anatomical structures of the status related 3D computer model and subsequently the graphical 3D sub-models of implants and/or surgical instruments of the 3D reference computer model are registered to the graphical 3D sub-models of implants and/or surgical instruments of the status related 3D computer model.

The generation of a graphical 3D computer model by using the 3D reference computer model and/or the pre-operative status related 3D computer model preferably comprises the step of: computer-aided planning and performing a virtual surgical treatment of anatomical structures to be treated.

In another embodiment the graphical 3D computer model comprises a graphical 3D sub-model of the anatomical structures to be treated in the form of a digital data set by using the first and second medical images.

In another embodiment the computer-aided planning comprises an integration of at least a further graphic 3D sub-model of an implant in the graphical 3D computer model.

In a further embodiment the computer-aided planning comprises an integration of at least a further graphic 3D sub-model of a temporary auxiliary means, preferably of a surgical instrument in the graphical 3D computer model. By this means the position of implants or temporary equipment, such as guide wires, surgical tools and instruments can be spatially determined and represented in each treatment step up to the end of the therapy. This is achieved by matching the positions of corresponding 3D computer models of implants or temporary auxiliary means which are archived in the computer and can be retrieved, with firstly the correctly positioned 3D computer models of anatomical structures (as described above) and secondly with the positions of the implants and/or temporary auxiliary means visible on the X-ray images. The 3D computer models of implants or temporary auxiliary means are thus represented spatially over the complete course of therapy by repeated registrations on the different imaging modalities such as conventional preoperative x-rays, intraoperative planar 2D or spatial 3D C-arm images, or postoperative X-ray images.

In a further embodiment the computer-aided planning comprises an assessment of the bio-mechanical stability of the virtually surgically treated anatomical structures using a computer simulation, preferably using a finite element computer analysis. By means of computer-aided analysis and planning of the surgical operation, i.e. the re-positioning of the anatomical structures, the type and position of temporary and permanent implants can be spatially represented, virtually planned on the computer and the biomechanical stability e.g. of an osteosynthesis can be assessed by means of computer simulation and re-evaluated in each treatment step. The treatment plan can then be continued or modified if necessary.

In again a further embodiment the graphical 3D computer model comprises at least a graphical 3D sub-model of at least an intermediate result of anatomical structures virtually treated according to the computer-aided planning.

In another embodiment the graphical 3D computer model comprises as a sub-model a treatment plan, which preferably defines the exact sequence of surgery and includes appropriate control requirements.

In a special embodiment of the method for monitoring a surgical treatment a status related 3D computer model is generated in a preoperative status allowing a monitoring of at least an object before surgical treatment.

In a further embodiment a status related 3D computer model is generated in at least one intraoperative status allowing a monitoring of the at least an object during surgical treatment.

In a further embodiment a status related 3D computer model is generated in at least one postoperative status allowing a monitoring of the at least an object after surgical treatment.

In another embodiment the method further comprises assessing and/or analysing differences between the 3D reference computer model and a status related 3D computer model.

In another embodiment the method further comprises assessing and/or analysing differences between the graphical 3D computer model and a status related 3D computer model.

In again another embodiment the method further comprises assessing and/or analysing differences between a status related 3D computer model and a subsequent status related 3D computer model.

In a further embodiment the differences are automatically assessed and/or analysed.

A preferred use of the method is for the quality assurance of surgical treatments. A further component and advantage of this method is that all data that is generated over the entire course of therapy can be integrated into a quality management system and can thus be analyzed. This can positively affect in turn the style, selection and implementation of the therapy; for example standardizing the therapy procedures with respect to relevant parameters.

Furthermore the method for generating a 3D reference model and/or the method for generating a status related 3D computer model and/or the method for generating a graphical 3D computer model and/or the method for monitoring a surgical treatment can be used for:
a treatment of bone fractures.
a treatment of osseous deformities.
for dental implantology.

### A BRIEF DESCRIPTION OF THE DRAWINGS

Several embodiments of the invention will be described in the following by way of example and with reference to the accompanying drawings in which:

FIG. 1 illustrates a lateral view of a patient's fractured bone; and

FIG. 2 illustrates a schematic view of a registration step according to an embodiment of the method according to the invention;

FIG. 3 illustrates a perspective view of a 3D reference computer model of the patient's bone in an unfractured state according to an embodiment of the method according to the invention;

FIG. 4 illustrates a flow chart of an embodiment of the method for generating a status related 3D computer model in a pre-operative status according to the invention;

FIG. 5 illustrates a flow chart of an embodiment of the method for generating a status related 3D computer model of a patient's anatomical structure in a pre-, intra- or post-operative status according to the invention; and

FIG. 6 illustrates a flow chart of an embodiment of the method for generating a 3D computer aided planned model according to the invention.

### DETAILED DESCRIPTION OF THE INVENTION

Image based assessment of a bone fracture/deformity is always based on the experience of the assessor and therefore restricted by his subjective interpretation. Standard two-dimensional (2D) imaging is commonly used for patient assessment providing only restricted information. The aim of the invention is an objective 3D assessment of the individual situation by providing a full 3D model of the individual clinical case based on standard images taken to assess the clinical situation.

Basically the method according to the invention can be applied for all anatomical structures, which can be acquired by means of a computer-aided medical imaging technique. Further, all implants and instruments that can be used intraoperatively and which are geometrically clearly detectable at least in part by a computer-aided medical imaging procedure can also be used.

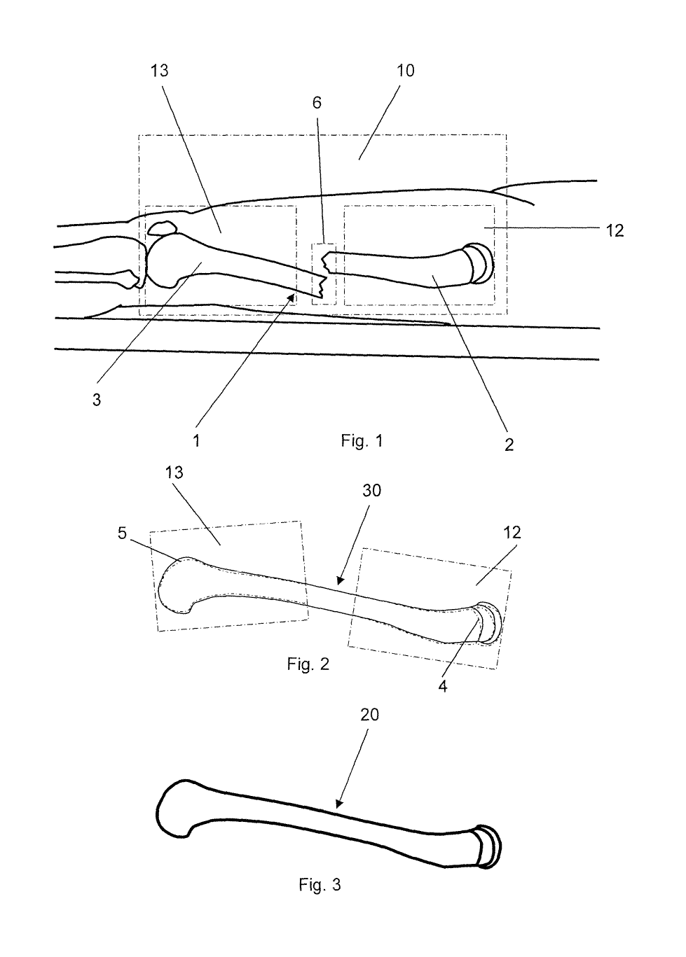

An exemplary embodiment of the method according to the invention for generating a 3D reference computer model 20 of a bone, and in particular of a femur is elucidated with reference to FIGS. 1 to 3. A full status related 3D computer model 25 of the individual clinical case based on at least a first and a second standard medical image 10, 11 is provided to assess the clinical situation.

Exemplarily, the method for generating this 3D reference computer model 20 of at least one anatomical structure comprises the steps of: A) acquiring at least a first and a second medical image 10, 11 of at least one anatomical structure in a preoperative status and from different perspectives by using a computer assisted medical imaging device, wherein the first and second medical images 10, 11 are represented by a respective first and second set of digital 2D image data; and B) generating a 3D reference computer model 20 of an anatomical structure by: i) selecting and extracting a 3D atlas model 30 of an anatomical structure to be treated from a generic anatomical atlas provided in the form of a digital data source; and ii) registering at least a section 12, 13 of each of the first and second medical images 10, 11 to the selected 3D atlas model 30. Preferably, the first and second medical images 10, 11 are taken from different perspectives that are minimum 60° angularly offset with respect to each other. The registering step can include before performing the image registration the sub-steps of: 1) extracting a first section 12 of the first medical image 10, wherein the first section 12 of the first medical image 10 comprises a section 4 of a proximal bone fragment 2 spaced apart from a fracture site 6 or from a deformed portion of a bone 1; 2) extracting a second section 13 of the first medical image 10, wherein the second section 13 of the first medical image 10 comprises a section 5 of a distal bone fragment 3 spaced apart from a fracture site (6) or from a deformed portion of a bone 1; and 3) repeating the above steps for the second medical image 11.

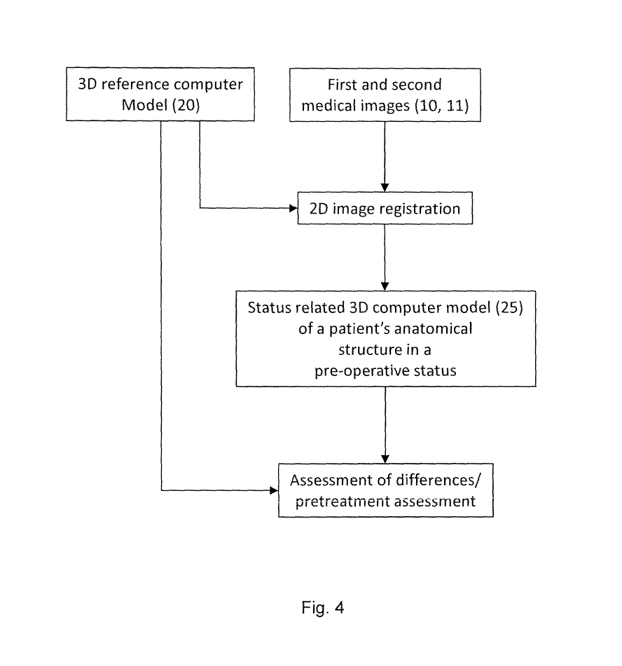

As illustrated in FIG. 4, this 3D reference computer model 20 of at least one anatomical structure can be used for comparison with the pre-operatively acquired first and second medical images 10, 11 of at least one anatomical structure or with a graphical 2D or D3 computer model 15 thereof.

By subsequently superposing this 3D reference computer model 20 on the first and second medical images 10, 11 additional variations especially from the pathological area are detected either as deformities or as fragments in dislocation. By using this technology this 3D reference computer model 20 as a full 3D model of the healthy situation can be compared with a corresponding status related 3D computer model 25, e.g. a pathological 3D model to assess the current situation at any stage of treatment. By this means a first pre-operative status related 3D computer model 25 of a patient's anatomical structure in the pre-operative status can be obtained by performing the step of registering each of the first and second medical images 10, 11 to the 3D reference computer model 20.

By comparing the 3D reference computer model 20 with the first pre-operative status related 3D computer model 25, which has been obtained by using the first and second medical image 10, 11, the actual situation of the at least one anatomical structure of a patient can be assessed and/or differences between the 3D reference model 20 and the first pre-operative status related 3D computer model 25 can be automatically and/or manually analyzed in order to characterize the clinical picture of the at least one anatomical structure of a patient.

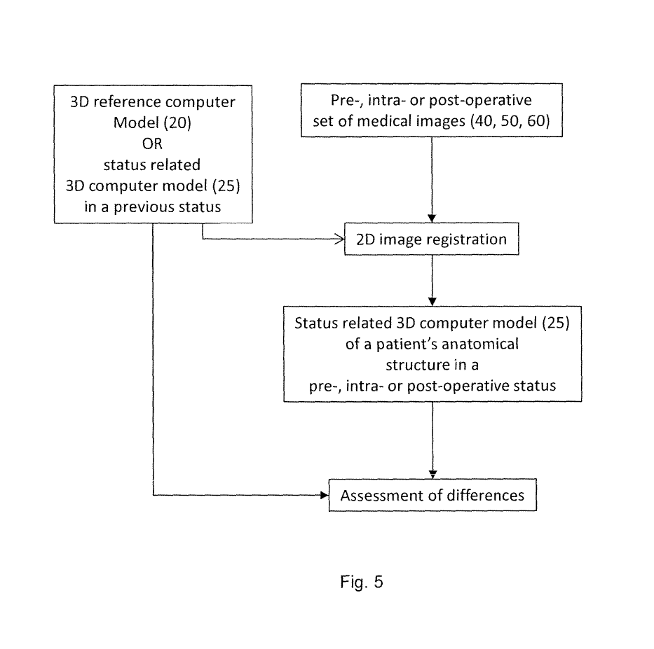

As illustrated in FIG. 5, the 3D reference computer model 20 of at least one anatomical structure can be used for comparison with a selectable pre-, intra- or postoperative set of medical images 40, 50, 60 of at least one anatomical structure or with a graphical 2D or D3 computer model 15 thereof. Status related 3D computer models 25 in a pre-, intra- or post-operative status can be obtained by performing the steps of: i) acquiring a pre-, intra- or post-operative set of medical images 40, 50, 60 including at least two medical images of at least one anatomical structure in a pre-, intra- or post-operative status and from different perspectives by using a computer assisted medical imaging device, wherein the at least two medical images are each represented by a respective set of digital 2D image data; ii) generating a graphical 2D or 3D computer model 15 of at least one anatomical structure in the form of a set of digital data by using the pre-, intra- or post-operative medical images 40, 50, 60; and iii) registering the graphical 2D or 3D computer model 15 to the 3D reference computer model 20.

By using these pre-, intra- or post-operative sets of medical images 40, 50, 60 the full tracking of treatment can be made. Alternatively, any kind of three-dimensional (3D) image information of a patient may be used to be compared either with the 3D atlas models 30 or with the 3D reference computer model 20, i.e. the 3D redesign of the individual healthy body as well as with any subsequent status related 3D computer model 25, i.e. with any pathological 3D redesign as captured with the method according to the invention at any stage of healing.

The 3D reference model 20 or any further post-operative status related 3D computer model 25 of a healthy situation (healthy 3D redesign) can be used to enhance the full body 3D atlas by adding its specific deviation values to the atlas or even by adding new specific values in the measured areas to the value maps when carefully validated. Using this loop the 3D atlas model 30 is automatically “learning” from any new information. If the 3D atlas model 30 would be available on the worldwide web to any system using this technology, all the systems would profit from a fast growing 3D atlas model allowing more and more precise assessments and the community of systems would learn to distinguish between “normal” as being in a certain range of variation in a certain set of patients as well as “pathological” being outside these variations in the healthy regions of the assessed patients.

### Example 1

Hereinafter, the method for generating a 3D reference computer model 20 according to the invention, the method for generating a status related 3D computer model 25 according to the invention and the method for generating a graphical 3D computer model 21 are described at an example of a surgical treatment of bone fractures and a correction of osseous deformities.

First, preoperative first and second medical images 10, 11 of the anatomical structures of a patient to be treated are acquired by means of a computer-aided medical imaging procedure. The method includes obtaining adequate image information of the operation area prior to surgery. The method provides acquiring a preoperative first medical image data set of an anatomical structure of a patient to be treated, preferably using a CT, for example the region with a bone fracture or osseous deformity. Alternatively, or in addition other 3D layer imaging techniques such as cone beam computed tomography can (called digital volume tomography), magnetic resonance tomography or 3D laser scanning can be used. As an output the first preoperative medical image data set will be obtained in the form of a digital image data set, for example, a data set in the DICOM format (digital imaging and communication in medicine).

Second, a 3D reference computer model 20 of the anatomical structures to be treated is generated as a digital data set by using the first and second medical images 10, 11. In particular, identification, localization and representation of the anatomical structures before the operation is effected in this step.

Using the preoperative first and second medical images 10, 11, the anatomical structures to be treated, e.g. bone fragments in the case of bone fractures or bone segments in the case of osseous deformities are identified, located and stored in the form of the 3D reference computer model 20 using appropriate computer software, so that the anatomical structures can represented e.g. as 3D bone fragments on a computer screen. This can be effected by methods of identification, i.e. the detection of anatomical geometric patterns of the anatomical structures such as bone fragments; their localization, i.e. the definition of their spatial location; and their representation, i.e. their adequate spatial representation as a 3D computer model. This includes also techniques of image segmentation. For example, in the case of corrective osteotomies two or more virtual bone fragments according to the osteotomy planning are identified and localized in this step, wherein a prospective cutting line is used to separate the bone fragments. This step is effected automatically or manually on a computer before the operation, wherein as input the preoperative first and second medical images 10, 11 and computer software and methods for the processing of this image data set are used, i.e. for the identification, localization and spatial representation of the 3D anatomical structures like e.g. bone fragments in the case of bone fractures. A processed digital set of data will be obtained as output, which permits a graphical representation of the anatomical structures, e.g. the individual bone fragments.

The 3D reference computer model 20 obtained as described above can be matched with respect to its spatial position by means of image registration with a status related 3D computer model 25 which can be generated from a pre-, intra- or postoperative set of digital medical images 40, 50, 60. Therewith, a status related 3D computer model 25 can represented on a computer screen in the actual pre-, intra- or postoperative position of the anatomical structures to be treated over the entire course of therapy. Within a monitoring of surgical treatment therefore the 3D reference computer model 20 can be used for a position-oriented representation of the anatomical structures to be treated, preoperatively in the operating room immediately before surgery, intraoperatively, after completing the surgery and/or post-operatively after surgery for the quality assurance of the surgical treatment as described below.

Before the registration step of the 3D reference computer model 20 with a status related 3D computer model 25 is effected, the desired pre-, intra- or postoperative set of medical images 40, 50, 60 of the anatomical structures to be treated and/or the implants is obtained by means of a computer-aided medical imaging.

This is followed by generating a status related 3D computer model 25 of the anatomical structures to be treated as a digital data set. After the digitized pre-, intra- or postoperative set of medical images 40, 50, 60 have been obtained, e.g. by using pre-intra- or postoperative X-ray imaging of the anatomical structures, the same anatomical landmarks of the anatomical structures, e.g. from bone fragments and bone contours of the fracture zone and the healthy bone surface including the articular surface, bone grey values and/or geometric bone patterns are re-identified and re-localized on one or more of the digitized medical images or directly on the status related 3D computer model 25, to subsequently register the 3D reference computer model 20 of the anatomical structures to be treated, e.g. the bone fragments to the status related 3D computer model 25 in the pre-, intra- or postoperative situation. Conventional planar X-rays, X-rays in two planes, or X-rays obtained in the operating room immediately prior to surgery, which have been preferably obtained by means of a 2D or 3D imaging process using a C-arm, are used as pre-, intra- or postoperative imaging techniques.

Subsequently, the registration of the 3D reference computer model 20 with the status related 3D computer model 25 is performed. A new representation is therefore achieved, wherein the 3D reference computer model 20 of the anatomical structures to be treated, e.g. the bone fragments position are visible in their correct position according to the actual pre-, intra- or postoperative medical imaging. Any shifts in the position of the anatomical structures, e.g. the bone fragments after the time of acquisition of the first and second medical images 10, 11 or a computed tomography (CT) are therefore actualized and thus compensated.

Alternatively, instead of using the 3D reference computer model 20 for registration with any status related 3D computer model 25 a graphical 3D computer model 21 that has been obtained by computer-aided planning can be used for registration with any status related 3D computer model 25. This graphical 3D computer model 21 can be generated by using the 3D reference computer model 20 and/or the pre-operative status related 3D computer model 25 as a basis and by further performing the step of computer-aided planning and/or performing a virtual surgical treatment of anatomical structures to be treated. Analogously to the generation of the 3D reference computer model 20 the generation of this graphical 3D computer model 21 comprises an identification, localization and representation of the anatomical structures prior to surgery.

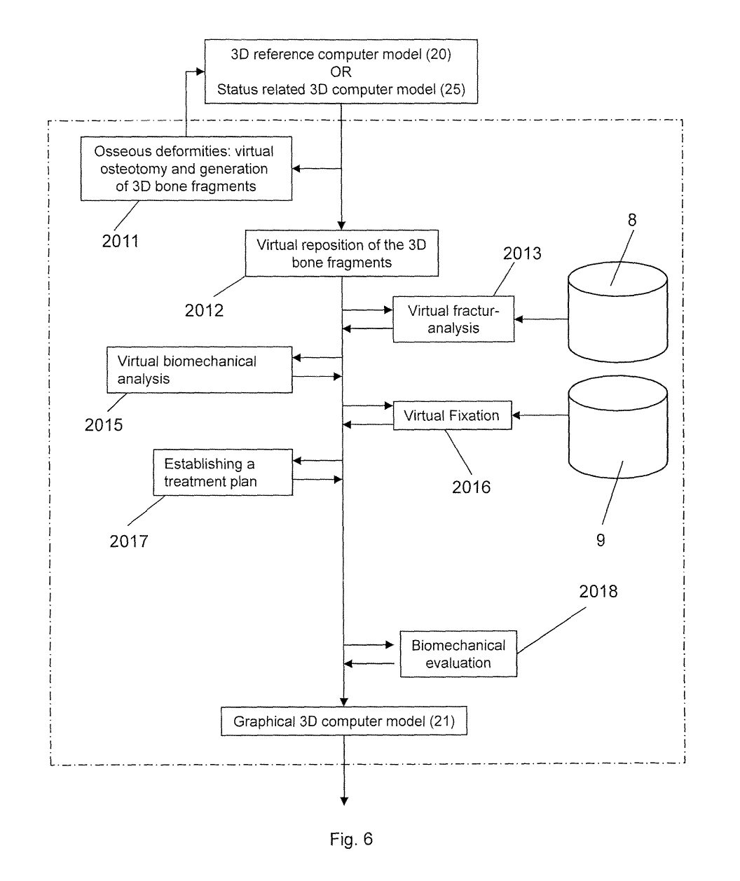

The 3D preoperative planning on the computer is represented in detail in FIG. 6, wherein the 3D preoperative planning on the computer may include all or only a part of the steps 2011 to 2021 represented in FIG. 6. In addition to the clinical examination of the patient, studies of the clinical documentation including assessment of the medical imaging now a preoperative planning of the surgical treatment is effected on the computer using appropriate software: herein, for example, the correct virtual reduction of the 3D bone fragments in the case of bone fractures is a central task (step 2012). The anatomical reduction of 3D bone fragments allows the representation and analysis of bone rest defects, if any. In the case of osseous deformities, however, the osteotomy is spatially set (step 2011) virtually on the computer and then the 3D bone fragments are moved in the planned position (step 2012). Thereto, the above defined 3D bone fragments are constantly newly represented, respectively registered according to the planned position of the osteotomy.

As a further feature of this 3D preoperative planning on the computer the fracture or the osteotomy can be virtually analyzed (step 2013). By this means shape, size and the degree of dislocation of bone fragments and the residual defect or the created defect, as well as resulting overlapping of bone fragments (important in the case of osteotomies or bone grafting) can be calculated. Furthermore, well-known fracture classifications 8, e.g. the classification of AO COIAC, or Müller AO classification, which are stored and available on databases, can be used.

Then, the virtual osteosynthesis (step 2016) for bone fractures as well as for osseous deformities is planned by selecting archived 3D computer models 9 temporary equipment, e.g. surgical instruments, and definitive implants like plates, intramedullary nails, screws, guide wires, in appropriate size and positioning the same in the graphical 3D computer model 21 as a graphical 3D sub models. In the case of bone defects the planning of autologous or alloplastic material (e.g. bone graft or cement) together with the quantity can be additionally included, wherein the defect is virtually restored with corresponding virtual packings, which correspond to the volume and the mechanical properties of bone. Furthermore, an execution plan (step 2017) is determined and integrated as a sub-model in the graphical 3D computer model 21, which defines the exact sequence of surgery and includes appropriate control requirements. By this means the sequence of the reduction of the bone fragments or osteotomies is determined, as well as the sequence and use the temporary tools and the definitive implants. A virtual graphic 3D computer model of the interim results that can be compared with the real intermediate result during the operation is part of the control requirements.

As a further feature of this 3D preoperative planning on the computer the virtual osteosynthesis consisting of bone fragments and implant which has been obtained during the operation planning can be virtually bio-mechanically tested (step 2018), e.g. by means of a finite element analysis.

As input the preoperative status related 3D computer model 25 is used. On this basis graphical 3D sub-models of bone fragments, respectively of the whole region with osseous deformities can be established prior to planning. The following software tools can be used for the planning and execution of a virtual surgical treatment:
1. Software tool for generating virtual osteotomies, particularly in the case of osseous deformities;
2. Software tool for virtual re-positioning of the 3D bone fragments;
3. Archived 3D computer templates of temporary auxiliary means and definitive implants like plates, screws, intramedullary nails, Kirschner wires;
4. Software tool for the analysis of the components (such as number, size, geometry of bone fragments and implants) and the planning processes (e.g. degree of dislocation, osteotomy angle) during the planning;
5. Software tool for establishing a primary execution plan and alternatives; and
6. Software tool for the analysis of the biomechanical properties of the osteosynthesis.

A graphical 3D computer model 21 is generated as output, which can include the anatomical structures virtually surgically treated in accordance with computer-based planning including the implants and/or surgical instruments, one or more graphical 3D sub-models of one or more intermediate results of the anatomical structures virtually treated according to the computer-based planning and a computer-based planning of osteosynthesis for treating fractures, respectively for the correction of osseous deformities.

The 3D monitoring of the surgical treatment can comprise one or more of the subsequently described steps:
1) Monitoring before the operation; and/or
2) Monitoring during the operation; and/or
3) Monitoring in the case of postoperative treatment control.

### 1. Monitoring Prior to Surgery:

At the beginning, a pre-operative set of medical images including a first and second medical image 10, 11 of anatomical structures to be treated is acquired. Anatomical landmarks of bone fragments and bone contours of the fracture zone and healthy bone surface including an articular surface, bone grey values as well as geometric patterns of bone are re-identified and re-located on the preoperative X-ray images to register the 3D reference computer model 20 to the pre-operative status related 3D computer model 25. Conventional planar X-ray or X-rays in two planes are used as preoperative imaging techniques, or X-rays acquired in the operating room immediately before the operation, preferably acquired by using a 2D or 3D C-arm.

As a result a new representation is achieved on which the pre-operative 3D computer model 25 of the anatomical structures, e.g. the bone fragments are visible in their correct position according to the actual imaging. Any location shifts of bone fragments from the time after the image acquisition of the first and second medical image 10, 11 can be updated accordingly and thus compensated.

Now, the 3D surgical planning can be included, i.e. the entire planned osteosynthesis construct can be visualized including the positions of implants and their insertion direction and end position. Thus, a prospective spatial positioning of implants is carried out pre-operatively as well. After registration of all the described components, the various components can demand shown on the computer or hidden.

### 2. Monitoring During the Operation:

A further X-ray control is effected, but now intraoperatively during surgery, preferably a 2D or 3D C-arm image control. As well a further image registration as described above is performed: so, anatomical landmarks of bone fragments and bone contours of the fracture zone and healthy bone surface including an articular surface, bone grey values as well as geometric patterns of bone are re-identified and re-located on the intraoperative X-ray to register the pre-operative status related 3D computer model 25 of bone fragments. The actual position of the 3D bone fragments can thus be spatially determined or monitored intraoperatively. If an implant is fixed to bone at the beginning of the operation the registration process can be improved or facilitated. This can be useful especially for corrective osteotomies, since less anatomical landmarks are at the disposal, which are identifiable analogously in the preoperative 3D imaging.

In the case of corrective osteotomies it can be useful to firstly effect only a partial shift to evaluate the spatial position the bone fragments by means of re-identification and re-localization. Further measures can be initiated to improve the result of the osteotomy. Only after control of the spatially correct position of the bone fragments the definitive fixation is performed.

Once implants and/or surgical instruments are visible on another intraoperative X-ray control in the course of the operation their spatial position can be determined by a registration with the previously spatially defined pre-operative status related 3D computer model 25 of the bone fragments and a corresponding positioning of graphical 3D sub-models of implants or surgical instruments.

Again a 3D surgical planning can be included, i.e. the planned and current osteosynthesis can be visualized, analysed and tested virtual bio-mechanically including positions of implants and/or surgical instruments and their direction of insertion and final position.

Further X-ray controls with repeated re-identification and re-localization during operation including information of the preoperative planning and simulation may assist the surgeon to continue the operation successfully and three-dimensionally documented, to modify and finally terminating with a control of the spatial location of the osteosynthesis.

### 3. Monitoring During Postoperative Controls of Progression

Routine post-operative controls of progression by means of X-ray controls are carried out. On these X-rays the status related 3D computer model 25 of the bone fragments as well as the graphical 3D sub-models of implants can be selectively re-identified and re-localized after the osteosynthesis. By means of the postoperative X-ray controls it can be determined whether or when a spatial position shift of bone fragments or the implants has occurred, in particular whether a shift has occurred postoperatively. Again the position of the pre-operative status related 3D computer model 25, i.e. of bone fragments and the implants can be compared with subsequent pre- or intra-operatively generated status related 3D computer models 25. The computerized preoperative planning can be visualized and the current situation can be simulated e.g. by means of finite element analysis in order to test the biomechanical stability of current osteosynthesis. In further controls of progression a re-evaluation can be performed, i.e. based on the results represented it can be decided whether the therapy can be terminated or whether new diagnostic or therapeutic measures should be initiated.

If a precise registration on a planar X-ray only can be achieved, the standard X-ray documentation “in two levels” is not necessary. Thus, the radiation exposure and expenditure can be reduced.

One or more of the findings and results obtained during steps effected during the monitoring procedure can be transferred into a quality management system for surgical treatments.

The method for monitoring a surgical treatment can be effected by comparing any status related 3D computer model 25 with the 3D reference model 20. Any status related 3D computer model 25 may be compared either with the 3D atlas models 30 or with the 3D reference computer model 20, i.e. the 3D redesign of the individual healthy body as well as with any previous status related 3D computer model 25, i.e. with any pathological 3D redesign as captured with the method according to the invention at any stage of healing.

Alternatively, instead of using the 3D reference computer model 20 for comparison with any status related 3D computer model 25 a graphical 3D computer model 21 that has been obtained by computer-aided planning can be used for comparison with any status related 3D computer model 25. This graphical 3D computer model 21 can be generated by using the 3D reference computer model 20 and/or the pre-operative status related 3D computer model 25 as a basis and by further performing the step of computer-aided planning and/or performing a virtual surgical treatment of anatomical structures to be treated. Analogously to the generation of the 3D reference computer model 20 the generation of this graphical 3D computer model 21 comprises an identification, localization and representation of the anatomical structures prior to surgery.

### Example 2

The method for generating a 3D reference computer model 20 according to the invention, the method for generating a status related 3D computer model 25 according to the invention and the method for generating a graphical 3D computer model 21 are described below at another example for applications in the dental implantology. The course of therapy in the case of implantation of one or more dental implants can be monitored over the course of the therapy as follows: preoperatively at least a first and second medical image 10, 11 of the operation area and the neighbouring region, e.g. around the adjacent teeth and/or of the alveolar ridge are acquired, i.e. a preoperative medical 3D image data set is obtained 10 and a 3D reference computer model 25 and/or a pre-operative status related 3D computer model 25 and/or a sub model thereof is generated. Preferably, the 3D imaging is performed using an optical 3D scanning procedure, e.g. laser scanning. This 3D imaging can be effected solely or in addition to a preoperative CT or digital volume tomography. The monitoring of the individual therapy steps is now effected by acquiring the surgical field before, and then during the surgery including the surgical instruments like pilot drills and the dental implants, as well as immediately after surgery or after introduction of the dental prosthetic work (i.e. a crown or bridge) by means of the optical laser scanning together with the neighbouring region, and by registering these 3D images obtained at various stages of therapy. The 3D images described form additional status related 3D computer models 25, which were generated on the basis of one or more pre-, intra- or postoperative sets of medical images and which have been registered with the 3D reference computer model 20. This registration should be preferably performed at non-operated structures, e.g. on anatomical structures such as teeth or the alveolar ridge. The registration allows the determination of the spatial position of the implants and surgical instruments. Steps including a 3D preoperative planning can be included in the therapy as described. The result of the therapy, e.g. the entire dental prosthetic treatment, can be compared with the virtual planning, respectively re-evaluated in any phase.

An advantage of this embodiment of the invention is that laser scanning is a 3D imaging modality without generating radiation. It can be used as soon as surfaces of the operation region as well as implants, surgical instruments, but also fracture segments and osteotomies are sufficiently visible and thus detectable. Advantageously, no additional exposure of the patient to radiation is required. A further advantage is the very detailed reproduction of surfaces like those of the teeth or implants.

Alternatively, conventional dental X-rays for monitoring over the course of the therapy can be used in the field of dental implantology, as described. Here, an X-ray exposure is present, but, however, minimal. If the implants or surgical instruments are not directly sufficiently visible, because they are located in the bone and/or under the mucous membrane, and thus cannot or insufficiently be acquired by means of laser scanning, temporary bodies with known geometry, e.g. an in-growing cap, can be screwed on the implants or surgical instruments. If the operated region with a well visible in-growing cap per inserted implant is now scanned, the corresponding computer template of the in-growing cap including the computer template of the inserted implant or surgical instrument can be included in the registration procedure so that their positions can be unambiguously determined.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1: A method for generating a 3D reference computer model of at least one anatomical structure for comparison with a selectable pre-, intra- or postoperative set of medical images of at least one anatomical structure, the method comprising:
acquiring at least a first and a second medical image of at least one anatomical structure in a preoperative status and from different perspectives using a computer assisted medical imaging device, wherein the first and second medical images are represented by a respective first and second set of digital 2D image data; and
generating the 3D reference computer model of the anatomical structure by:
selecting a 3D atlas model of an anatomical structure to be treated from a generic anatomical atlas provided in the form of a digital data source; and
registering at least a section of each of the first and second medical images to the selected 3D atlas model.

2: The method according to claim 1, wherein the first and the second medical images are taken from different perspectives that are a minimum of 60° angularly offset with respect to each other.

3: The method according to claim 1, wherein the at least one anatomical structure is a bone, and wherein the method further comprises:
extracting a first section of the first medical image, wherein the first section of the first medical image comprises a section of a proximal bone fragment spaced apart from a fracture site or from a deformed portion of a bone;
extracting a second section of the first medical image, wherein the second section of the first medical image comprises a section of a distal bone fragment spaced apart from a fracture site or from a deformed portion of a bone; and
repeating the above steps for the second medical image.

4: The method according to claim 1, wherein the first and second medical images include a plurality of anatomical structures and the 3D reference computer model comprises a graphical 3D sub-model for each anatomical structure.

5: The method according to claim 1, further comprising:
introducing at least one digital graphical 3D sub-model in the 3D reference computer model.

6: The method according to claim 5, wherein the digital graphical 3D sub-model represents an implant.

7: The method according to claim 5, wherein the digital graphical 3D sub-model represents a surgical instrument.

8: The method according to claim 1, wherein the generation of the 3D reference computer model comprises an automatic or manual identification and localization of anatomical landmarks, lines and/or regions of the anatomical structures to be treated.

9: The method according to claim 6, wherein generation of the 3D reference computer model comprises an automatic or manual identification and localization of distinctive points, lines and/or regions of the implant.

10: A method for generating a status related 3D computer model of a patient's anatomical structure in a pre-operative status by using a 3D reference computer model generated according to claim 1, the method comprising:
registering each of the first and second medical images to the 3D reference computer model.

11: A method for generating a status related 2D or 3D computer model of a patient's anatomical structure in a pre-, intra- or post-operative status by using a 3D reference computer model generated according to claim 1, the method comprising:
acquiring a pre-, intra- or post-operative set of medical images including at least two medical images of at least one anatomical structure in a pre-, intra- or post-operative status and from different perspectives by using a computer assisted medical imaging device, wherein the at least two medical images are each represented by a respective set of digital 2D image data;
generating a graphical 2D or 3D computer model of at least one anatomical structure in the form of a set of digital data by using the pre-, intra- or post-operative medical images; and
registering the graphical 2D or 3D computer model to the 3D reference computer model.

12: The method according to claim 10, wherein the status related 3D computer model additionally comprises a representation of at least one implant.

13: The method according to claim 10, wherein the status related 3D computer model additionally comprises a representation of at least one surgical instrument.

14: The method according to claim 10, wherein the pre-, intra- or postoperative set of medical images includes a plurality of anatomical structures and the status related the 3D computer model comprises each a graphical 2D or 3D sub-model for each anatomical structure and preferably for each implant and/or surgical instrument.

15: The method according to claim 10, wherein during the registration step the status related 3D computer model forms the reference model to which the 3D reference computer model is adapted.

16: The method according to claim 10, wherein the acquisition of the set of medical images in the pre-, intra- or postoperative status includes an acquisition of one or more digitized medical images by means of a computer-aided medical imaging technique.

17: The method according to claim 10, wherein the generation of the status related 3D computer model includes an automatic or manual re-identification and re-localization of anatomical landmarks, lines and/or regions of the anatomical structures to be treated as identified and localized in the 3D reference computer model.

18: The method according to claim 10, wherein the generation of the status related 3D computer model includes an automatic or manual re-identification and re-localization of distinctive points, lines and/or regions of each implant and each surgical instrument as identified and localized in the 3D reference computer model.

19: A method for generating a graphical 3D computer model by using the 3D reference computer model generated according to claim 1, the method further comprising:
computer-aided planning and performing a virtual surgical treatment of anatomical structures to be treated.

20: The method according to claim 19, wherein the graphical 3D computer model comprises a graphical 3D sub-model of the anatomical structures to be treated in the form of a digital data set by using the first and second medical images.

21-37. (canceled)
